(19) United States
(12) Patent Application Publication
(10) Pub. No.: US 2016/0000851 A1
(43) Pub. Date: Jan. 7, 2016

(54) Bioactive Botanical Compositions and Uses Thereof

(71) Applicant: AKZO NOBEL CHEMICALS INTERNATIONAL B.V., Amersfoort (NL)

(72) Inventors: Michael Koganov, White Plains, NY (US); Li Zhang, Princeton, NJ (US); Artyom Duev, White Plains, NY (US)

(21) Appl. No.: 14/768,817

(22) PCT Filed: Mar. 10, 2014
(86) PCT No.: PCT/EP2014/054514

§ 371 Date: Aug. 19, 2015

Related U.S. Application Data
(60) Provisional application No. 61/840,711, filed on Jun. 28, 2013.
(60) Provisional application No. 61/779,450, filed on Mar. 13, 2013.

(51) Int. Cl. A61K 36/82; A61Q 19/10; A61K 9/00; A61K 8/97; A61K 36/752; A61K 36/48

(52) U.S. Cl. CPC A61K 36/82; A61K 36/752; A61K 36/48; A61K 9/0014; A61K 8/97; A61Q 19/10; A61K 2800/75

(57) ABSTRACT

The present invention relates to a composition having enhanced anti-inflammatory properties and to a method for inhibiting inflammation in biological tissue, including but not limited to skin Skin inflammation includes any undesirable effect produced in or on the surface of skin, including but not limited to irritation, redness, swelling, local temperature elevation, fissures, desquamation, itch, pain, sensitivity, abrasion, discoloration, and bleeding or the like, and combinations thereof. The invention demonstrates that certain plant fractions, such as the serum fractions of Camellia sinensis (Recentia® CS), Citrus limon (Recentia® CL), and Trifolium pratense (Recentia® TP), can be effectively utilized in various products to inhibit inflammation of biological tissue, including but not limited to skin. In one embodiment, said plant fractions have not undergone any significant fermentation.

### FIELD OF THE INVENTION

The present invention relates to compositions and methods for mitigating inflammation and irritation induced by surface active compounds.

### BACKGROUND OF THE INVENTION

Surface Active Compounds are substances that can decrease the surface tension or interfacial tension of a liquid when dissolved in it. Most commonly this refers to water and its interfaces with air, solid surfaces, and other substances. While any solute can change the properties of a solution, certain compounds especially effective at this are called surfactants. This effectiveness is the result of an amphiphilic structure that includes a hydrophobic part, commonly comprised of one or two hydrocarbon “tails”; and hydrophilic “head” part, which may be negatively charged (anionic surfactants), positively charged (cationic surfactants), lack a charge (nonionic surfactants), or have both positively and negatively charged groups within head structure (amphoteric or zwitterionic surfactants).

Some examples of surfactants belonging to these groups would be:
Anionic
   Sodium Dodecyl Sulfate (SDS)
   Sodium Olefin Sulphonate
Cationic
   Cetrimonium Bromide (or hexadecyltrimethylammonium bromide)
   Mono Alkyl Quat
   C12-C18 Ethoxylated Amine
Nonionic
   Octyl Phenol Ethoxylate
   Ethoxylated Alcohol
Zwitterionic/Amphoteric
   Cocamidopropyl betaine
   Coco Ampho Poly Carboxyglycinate

Amphiphilic structure allows surfactant molecules to aggregate and orient themselves at the interfacial boundaries, form complex arrangements, increase the wetting ability of solutions, allow suspension of hydrophobic substances, and interact with arrangements formed by other amphiphilic substances.

A biologically important example of such an arrangement is the plasma membrane of a living cell and organelles, which is a bi-layer structure formed by two layers of oriented phospholipids, with inner side of the membrane being the hydrophobic “tails” facing each other and outer sides facing the intracellular and extracellular medium being the hydrophilic “heads”. Other components important for cell function are embedded in or attached to the plasma membrane.

Health and viability of living tissues strongly depends on properties of the surrounding medium and on integrity of the plasma membranes of the cells of the tissue.

Introduction of a surfactant can alter the properties of the medium such as surface tension and impact the plasma membrane stability by increasing its permeability, or otherwise damaging it. Introduced surfactants can also interact with other plasma membrane components. Undesirable effects of surfactants on living tissues can cause complex biological responses such as inflammation and irritation.

In the past, many attempts have been made to reduce surfactant-induced skin irritation and inflammation by selecting less-irritating surfactants, blocking skin contact by occlusive films, reducing the critical micelle concentration of detergent to limit the exposure of skin to free surfactant monomers, or adding enzymes and conventional plant extracts to promote skin exfoliation and skin cell renewal. However, surfactant-induced skin irritation and inflammation are still one of the major concerns of the consumers. As surfactants have a great variety of industrial, scientific and household uses to allow or improve processes of wetting, emulsifying and solubilizing, cleaning, foaming, and dispersing.

Therefore, human contact with surfactants is a frequent occurrence, and mitigating harmful effects of such contact is desirable.

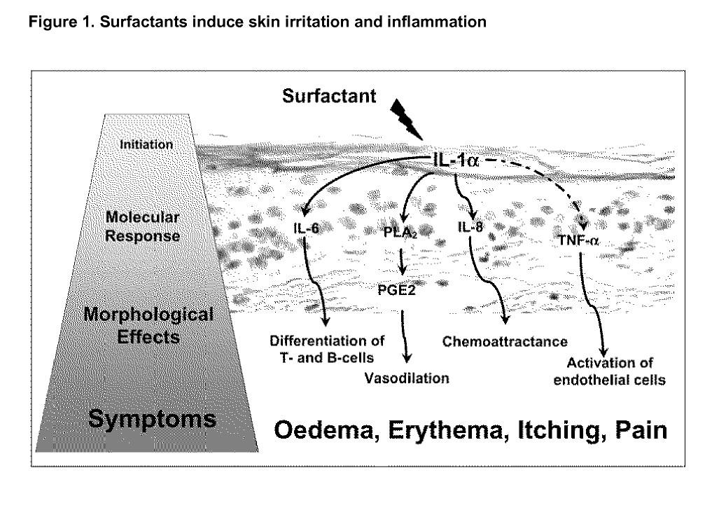

Inflammation is a complex cascade of biological reactions mediated by signaling substances including, but not limited to vasoactive amines such as histamine, products of arachidonic acid metabolism such as prostaglandins, and signaling proteins such as chemokines and interleukins in particular. Certain signaling molecules are particularly important in regulating the inflammation and quantification of inflammatory activity due to factors including but not limited to their position in inflammatory signaling cascades, broadness of their range of pro-inflammatory effects, and comparative efficacy at triggering the inflammatory responses. An example of such a cascade is provided in FIG. 1.

One of the methods of studying and quantifying such inflammation is by culturing cells of the tissue most likely to come in contact with surfactants, such as viable keratinocytes from the epidermis. These cultured cells could be subjected to stresses and treatments, and levels of released substances associated with inflammatory signaling or cellular damage could be determined by means of various bioassays. The skin keratinocytes have become the focus of attention in irritant-induced skin inflammation by virtue of their epidermal location, their importance in maintaining the integrity of the stratum corneum barrier, and their ability to produce a range of inflammatory mediators. Keratinocytes contain large quantities of biologically active Interleukin (IL)-1α, which can be released in response to a range of irritants, such as surfactants. IL-1α is one of the primary cytokines, which can be induced by irritants, and is often released from keratinocytes at the early stage of inflammation cascade. Subsequently, IL-1α leads to the induction of numerous down-stream inflammatory mediators, e.g. signaling molecules, cytokines, and chemokine IL-8, which is known as neutrophil chemotactic factor. IL-8 is important for the recruitment of leukocytes to damaged skin and for the development of the signs of skin inflammation. Therefore, by reducing secretion of IL-1α and IL-8 from keratinocytes, an initial inflammatory response mediator and a key chemotactic factor, the signs of the skin inflammation may be reduced, prevented, and/or eliminated.

Certain bioactive compositions (i.e. fractions) produced by process described, for example, in U.S. Pat. Nos. 7,442,391; 7,473,435; 7,537,791; 8,043,635; 8,101,212; 8,277,852 and 8,318,220 have compositions notably different from conventional solvent-extracted botanical extracts. Certain fractions have potent anti-inflammatory, anti-oxidant and photo-stabilization activities that may influence multiple biological pathways responsible for skin aging, while also minimizing deterioration of formulation stability, color and odor, which would make them especially suitable for topical applications. Suitable bioactive fractions can include, without limitation, a cell walls fraction, a cell walls fraction extract, a membrane fraction, a membrane fraction extract, a cytoplasm fraction, a cytoplasm fraction extract, a cell juice serum, and/or combinations.

### SUMMARY OF THE INVENTION

The present invention relates to methods for inhibiting inflammation in biological tissue, including but not limited to skin. Skin inflammation includes any undesirable effect produced in or on the surface of skin, including but not limited to irritation, redness, swelling, local temperature elevation, fissures, desquamation, itch, pain, sensitivity, abrasion, discoloration, and bleeding or the like, and combinations thereof. The present inventors have shown that the anti-inflammatory activity of certain plant fractions, such as the serum fractions of Camellia sinensis (Recentia® CS), Citrus limon (Recentia® CL), and Trifolium pratense (Recentia® TP), can be effectively utilized in various products to inhibit inflammation of biological tissue, including but not limited to skin.

### DETAILED DESCRIPTION OF THE INVENTION

The present invention relates to methods for inhibiting inflammation in biological tissue, including but not limited to skin, by contacting same with an effective amount of certain plant fractions, such as the serum fractions of Camellia sinensis (Recentia® CS), Citrus limon (Recentia® CL), and Trifolium pratense (Recentia® TP).

The present invention also relates to biomarkers of inflammation. In one embodiment, biomarkers of mammalian inflammation include, without limitation, biomarkers that are associated with Interleukin-1 alpha (IL-1α) inflammation cascades. IL-1α is an inflammatory cytokine, which is induced by irritants, and is often released from epidermal skin cells at the early stage of inflammation cascade. Subsequently, it leads to the induction of down-stream secondary inflammatory mediators including chemokine IL-8, followed by morphological alterations and finally the development of signs of skin inflammation. Therefore, by reducing secretion of IL-1α and IL-8, an initial inflammatory response mediator and a key chemotactic factor, skin inflammation and irritation can be reduced, prevented, and/or eliminated.

Certain surface active compounds have been found to induce skin inflammation and irritation. Surface active compounds, sometimes referred to as surfactants, are typically used in products to lower the surface tension of a liquid, the interfacial tension between two liquids, or that between a liquid and a solid. Surfactants may also act as a detergent, wetting agent, emulsifier, foaming agent, and/or dispersant. Surfactants can be anionic, cationic, nonionic, and zwitterionic surfactants and/or combinations thereof.

The present invention also relates to bioactive compositions. In one embodiment, the bioactive compositions include plant-derived isolated biologically active complexes produced by a process described in, for example, U.S. Patent Nos. 7,442,391; 7,473,435; 7,537,791; 8,043,635; 8,101,212; 8,277,852 and 8,318,220. These compositions, i.e. fractions, are not produced by conventional solvent extractions and their compositions are significantly different from the conventional botanical extracts. Suitable bioactive fractions can include, without limitation, a cell walls fraction, a cell walls fraction extract, a membrane fraction, a membrane fraction extract, a cytoplasm fraction, a cytoplasm fraction extract, a cell juice serum fraction, and/or combinations thereof.

The present invention also relates to bioactive topical formulations suitable for topical application to a mammal. In one embodiment, the bioactive topical formulations include topically effective amount of the bioactive compositions of the present invention. The bioactive topical formulations can further include topically acceptable carriers.

The present invention also relates to methods for inhibiting inflammatory activity in skin tissue of a mammal, including inflammatory activity in skin tissue caused by contacting the skin with one or more surfactants. This method involves providing the bioactive compositions according to the present invention. The method further involves applying the bioactive compositions to the skin tissue in amount effective to inhibit inflammatory activity in the skin tissue.

The present invention also relates to methods of protecting skin tissue of a mammal from surface active compound-induced damage. These methods involve providing the bioactive compositions of the present invention. The methods further involve applying the bioactive compositions to the skin tissue in an amount effective to reduce surface active compound-induced damage of the skin tissue and to prevent inflammatory damage of the skin tissue.

The present invention also addresses the deficiencies of conventional plant extracts, i.e., those produced by conventional methods. Conventional plant processing is deficient in that it fails to adequately preserve a broad spectrum of potent bioactive compositions. Processing of fresh Camellia sinensis, Citrus limon, and Trifolium pretense, etc. by process described in, for example, U.S. Pat. Nos. 7,442,391; 7,473,435; 7,537,791; 8,043,635; 8,101,212; 8,277,852 and 8,318,220, which are all incorporated herein by reference, without fermentation and excessive heat treatment unexpectedly demonstrates more powerful mitigation of surface active compound-induced inflammatory cytokine and chemokine secretions from epidermal skin cells than products of conventional plant processing.

The processes described in, for example, U.S. Pat. Nos. 7,442,391; 7,473,435; 7,537,791; 8043635, 8,101,212; 8,277,852 and 8,318,220, all incorporated herein by reference, uniquely preserves biological activities in natural ingredients. The plant fractions derived from these processes are also extremely effective in mitigating surfactant-induced secretion of IL-1α and/or IL-8 in epidermal skin cells. This was demonstrated by first inducing inflammatory cytokine IL-1α and/or IL-8 by different classes of surfactants, such as anionic, nonionic, cationic and zwitterionic, in cultured human epidermal keratinocytes. Then, the mildness of six representative surfactants from different classes were ranked based on their abilities to induce IL-1α and cytotoxicity. A nonionic ethoxylated alcohol, the mildest among the tested surfactants, was able to induce IL-1α but not IL-8. Next, serum fractions of Camellia sinensis (Recentia® CS), Citrus limon (Recentia® CL), and Trifolium pratense (Recentia® TP) were evaluated for inhibiting IL-1α and/or IL-8 in keratinocytes treated with either SDS or ethoxylated alcohol, and were compared to two well-known anti-inflammatory benchmark agents Aspirin and SB203580. The anti-inflammatory activity of products of the process, such as serum fraction of Camellia sinensis (i.e. Recentia® CS), was compared to that of Aspirin to show equal or better potency for inhibition of IL-1α induced by SDS and ethoxylated alcohol. In addition, both Citrus limon (Recentia® CL) and Trifolium pratense (Recentia® TP) also inhibited SDS-induced IL-1α to a lesser extent. When tested for mitigation of chemokine IL-8, Recentia® CS inhibited both SDS-induced and basal level IL-8 in keratinocytes, while SB203580 reduced SDS-induced IL-8 only. Finally, conventional green tea and black tea preparations obtained from the same cultivar as Recentia® CS failed to inhibit IL-1α and/or IL-8 induced by SDS and ethoxylated alcohol. Accordingly, the invention describes a novel approach to mitigate surfactant-induced skin inflammation and irritation by use of bioactive compositions produced by a process described, for example, in U.S. Pat. Nos. 7,442,391; 7,473,435; 7,537,791; 8,043,635; 8,101,212; 8,277,852 and 8,318,220.

### General Process Description

The process for the preparation of botanical fractions from fresh plant biomass and to compositions made from said fractions is exemplified as follows. The process comprises grinding (or maceration) and pressing fresh plant biomass in order to obtain an intracellular plant material (or plant cell juice) containing membrane fractions (containing nucleus, or chloroplasts, or chromoplasts, or mitochondria, or combinations of thereof), and treating said cell juice with electromagnetic waves at a frequency and for a time effective to trigger separation of said membrane fraction from said cell juice in order to yield a cell cytoplasm/cytosole fraction (all residual components of cell juice) substantially-free from membrane fractions. The aforementioned treatment is advantageously performed such that the temperature of said cell juice during said treatment does not exceed 40° C.

The botanical fractions derived from either the membrane fraction or the cytoplasm/cytosole fraction of fresh plants are unique both compositionally and in their activity. More specifically, the process described herein uniquely preserves anti-inflammatory, anti-oxidant, and other biological activities in natural ingredients. The plant fractions derived from these processes are also extremely effective in mitigating surfactant-induced secretion of IL-1α in epidermal skin cells.

The membrane fraction can then be utilized in order to provide a stable botanical cosmetic composition exhibiting antiproteolytic, cell growth inhibition activity, and/or both antiproteolytic and cell growth inhibition activities, where the antiproteolytic activity is due to inhibition of at least one proteinase and the cell growth inhibition activity is due to inhibition of cell growth of at least one type of cell.

The cytoplasm/cytosole fraction can be utilized in order to provide a botanical composition suitable for use as a component in a pharmaceutical, cosmetic, nutritional, therapeutic and/or personal care formulation and the like.

### Overall Process for Preparing Botanical Fractions of the Invention

The overall process for preparing the bioactive botanical cosmetic compositions of the present invention is described below. Fresh plants are harvested, collected, and washed to yield fresh plant biomass. This fresh plant biomass is subjected to grinding, maceration, and pressing to yield intracellular plant material (cell juice) and fiber-enriched material (press-cake). Cell juice is then filtered through nylon mesh to yield filtered plant cell juice. Filtered cell juice is exposed to electromagnetic waves treatment at a frequency to trigger its destabilization. Typically, the cell juice is subjected to an electromagnetic field at a frequency of 2.45 GHz, or the frequency of a conventional microwave. In another embodiment, the frequency of the electromagnetic field is greater than 2.45 GHz to about 7.0 GHz, or any individual frequency, or range of frequencies in between the range of 2.45 GHz and 7.0 GHz, in another embodiment from 2.5 GHz to 7.0 GHz, and in another embodiment from 3.0 to 6.0 GHz.

The destabilized cell juice is and then subjected to centrifugation in order to yield precipitated membrane fraction and a supernatant which is cytoplasm/cytosole fraction. Membrane fraction is a bioactive botanical cosmetic composition which can be added into various cosmetic products. Plant cytoplasm/cytosole fraction is used for further processes, as described below.

Cytoplasm/cytosole fraction can optionally be subjected to additional treatments: i, ii, iii or iv. as summarized below. As a nonlimiting example, treatment (i) can include isoelectric precipitation and following centrifugation enabling to separate precipitated cytoplasm fraction from supernatant containing cytosole fraction. Alternatively cytosole/cytoplasm fraction can be further separated as result of (ii) additional electromagnetic treatment with following centrifugation or filtration, or (iii) membrane filtration, or (iv) ultrafiltration, or combination of thereof (i, ii, iii, iv). Cytoplasm/cytosole fraction components can be utilized “as is” or can be further separated and utilized. They can also be stabilized with preservatives and antioxidants as described for example, in U.S. Pat. Nos. 7,442,391; 7,473,435; 7,537,791; 8,043,635; 8,101,212; and 8,277,852.

### Process for Preparing the Membrane-Derived Cosmetic Compositions

In one embodiment, the process for preparing the Membrane-Derived Cosmetic Compositions is as follows. This method involves providing plant cell juice that has been separated from a fresh plant biomass. “Fresh plant biomass” as it is used throughout this application is intended to mean that a majority of the freshly harvested plant biomass is in the living state and/or it has not undergone a meaningful amount of unwanted degradation. The plant cell juice is then treated under conditions effective to trigger separation it into a membrane fraction and a cell juice supernatant. The resulting membrane fraction has antiproteolytic activity, cell growth inhibition activity, or both antiproteolytic and cell growth inhibition activities. The membrane fraction is then converted under conditions effective to yield a stable bioactive botanical cosmetic composition exhibiting modulation of proteolytic, cell growth inhibition activity, or both proteolytic and cell growth inhibition activities, where the proteolytic activity is due to modulation of at least one proteinase and the cell growth modulation activity is due to modulation of cell growth of at least one type of cell.

The plant cell juice may be separated from all types of plants. Examples of suitable plants that may be used as sources of fresh plant biomass in the present include, without limitation, plants from the following families: Laminariaceae, Cladophoraceae, Fabaceae, Theaceae, Asteraceae, Lamiaceae, Liliaceae, Poaceae and Moraceae. In particular, examples of specific plants that have been tested and found appropriate as fresh plant biomass sources include Macrocystis pyrifera, Chaetomorpha basiretorsa, Medicago sativa, Trifolium pretense, Citrus limon, Glycine max, Camellia sinensis, Calendula officinalis, Tanacetum parthenium, Chamomilla recutita, Lavandula angustifolia, Salvia officinalis, Nelumbo nucifera, Lilium bulbiferum, Avena sativa and Hordeum vulgare. Various parts of the plants may be used. For example, the stems and leaf tissue may be used for many types of plants. For other plants, the flowers may be used as sources of plant cell juice for use in the present invention. For example, one embodiment of the present invention uses flower tissue of Calendula officinalis for the separation of the plant cell juice. In another embodiment, the leaf and stem tissue of Salvia officinalis is used.

The separation technique of the present invention allows one to isolate plant cell juice in a manner that preserves the bioactive components of the plant.

An exemplary method of preparing the plant biomass for use in extraction of plant cell juice involves harvesting, collecting, and washing of the fresh plants. Suitable steps to follow for preparing the fresh plant biomass include, for example, the following: (1) preservation of the inherent moisture content of the plant cells; (2) optimization of the height of cut used during harvesting of above-ground plant tissue; (3) reservation of plant integrity during harvesting (e.g., during cutting of the above-ground plant tissue); (4) minimization of environmental impact and time factors of biological degradation of the plant biomass; and (5) cleaning of the plant biomass prior to processing (e.g., prior to grinding and maceration). Each of these steps is discussed below.

### Preservation of Inherent Moisture Content:

The cutting should be done to avoid wilting due to moisture loss. Optimal conditions are those where natural moisture content is maintained and preserved.

### Optimal and Preferred Height of Cut:

The plants should be cut at least several centimeters above the ground to limit the amount of soil and other debris in the collected biomass. For example, all useable leaf and stem biomass of any given plant source may be cut at a height of greater than or equal to 5 centimeters above ground. If flower tissue is used as the plant biomass source, the flowers are separated from the whole plant prior to extraction of the plant cell juice.

### Preservation of Plant Integrity During Harvesting:

Harvesting of the plant biomass may be by cutting the above ground stem and leaf tissue of the plant. The cutting is conducted in a manner that avoids or minimizes the chopping, mashing, crushing, or other type of injury of the plant. For large-scale industrial harvesting, where it may not be possible to avoid chopping due to the type of equipment required, care is taken to minimize injury that could lead to microbial growth, moisture loss, intensification of oxidation, polymerization, isomerization, and hydrolysis processes (i.e., unwanted catabolic processes) in collected plants. For example, in one embodiment of the present invention, plants are cut and collected by hand as whole plants. In another embodiment, plant tissue is cut using harvesting equipment. In that case, the minimum chopping height above ground for each plant is greater than or equal to 5 centimeters. Further, particular attention is made to minimize injury during and after cutting. In another embodiment, flowering whole plants are collected by hand and the flowers are then separated for further processing.

### Minimization of Environmental Impact and Time Factors of Degradation:

Delivery time of cut plant material to the processing facility and exposure of biomass to sun, high temperature, and other negative environmental factors, should be minimized to prevent the impact of unwanted degradation processes as described above. For example, in one embodiment of the present invention, the delivery time for Fabaceae plants for further processing does not exceed 30 minutes from the time of cutting. In another embodiment, plants that undergo long distance transport are treated to a post-cutting procedure involving immediately placing the plant biomass into Styrofoam coolers containing bags of frozen gel packs to help maintain freshness and natural moisture content during overnight delivery to the processing facility. These procedures were conducted for plant biomass from Lamiaceae and Moraceae families. Other post-cutting procedures that achieve the results described above may be used as well. As a nonlimiting example, for many plant species it is beneficial to not only minimize delivery time for processing, but to also keep the cut plant material cool, by refrigeration if necessary, to prevent and/or minimize unwanted degradation prior to and/or during processing.

### Cleaning Step Prior to Grinding and Maceration:

A washing step to remove the soil particles and other debris from plants prior to further processing is performed once the plant tissue is harvested. The washing is achieved using a low-pressure rinse for a short duration under conditions to prevent the initiation of the release of the cell juice from biomass, to cause injury, or to remove valuable components. For example, in one embodiment of the present invention, the washing of the plant biomass was accomplished in less than or equal to 5 minutes with a water pressure of less than or equal to 1 kg/cm². Residual water wash did not contain any green or yellow pigments, which indicates the absence of subsequent injury. The excess water is removed from washed biomass in order to keep the dry matter content close to natural level.

After the plant tissue biomass is harvested, as described above, further processing of the plant tissue biomass is performed to yield plant cell juice. In one embodiment, the harvested plant tissue biomass is subjected to grinding, maceration, and pressing to separate the intracellular content, i.e., the cell juice, and to separate it from the fiber-enriched press-cake containing predominantly cell walls.

An example of a suitable processing protocol involves the steps described below. A hammer mill may be used to grind plants to yield plant tissue particles of a small size in a short time and without significant increase of biomass temperature. In one embodiment, a modified hammer mill is used to produce the maximum size of macerated plant particles less than or equal to 0.5 centimeters during less than or equal to 10 seconds of treatment, where the increase of biomass temperature is less than or equal to 5° C.

Exposure of ground and macerated plant biomass is minimized to prevent the impact of unwanted catabolic processes, as described above. The separation of plant cell juice from fiber-enriched material (or press-cake) is commenced as soon as possible after grinding and maceration of the plant biomass. The plant biomass is processed in a short time and without significant increase in temperature. In one embodiment, immediately after grinding and maceration, the plant biomass is pressed using a horizontal, continuous screw press (Compact Press “CP-6”, Vincent Corporation, Fla.). The pressure on the cone is maintained at level 24 kg/cm², screw speed is at 12 rpm, and biomass temperature increase is less than or equal to 5° C.

The initial cell juice usually contains small fiber particles, which can absorb valuable cell juice components and also block the hoses and pumps. The above particles should be removed by filtration or low-speed centrifugation. For example, the initial cell juices produced after the pressing step are filtered through four layers of nylon fabric prior to using the plant cell juice in the methods of the present invention.

Once plant cell juice is separated, the plant cell juice is relatively stable colloidal dispersion in which organelles represent the dispersed phase and cytoplasm represents the continuous phase. Cell juice is then treated to a processes involving (1) triggering destabilization of above colloidal dispersion performing a “initiation of membrane fraction aggregation step” to yield a destabilized cell juice and (2) performing a “membrane fraction separation step” on destabilized cell juice mixture to yield a membrane fraction (containing nucleous, or chloroplasts, or chromoplasts, or mitochondria, or combination of thereof) and a cell juice supernatant. In one embodiment, initiation of membrane fraction destabilization is accomplished by subjecting said cell juice to electromagnetic waves at a frequency of 2.45 GHz. In another embodiment the frequency employed is greater than 2.45 GHZ up to 7.0 GHz. After destabilization is achieved, a membrane fraction separation step is performed. This step includes, for example, separating of destabilized cell juice into the membrane fraction and the cell juice supernatant using separating techniques including filtration, or centrifugation, or combination of thereof.

A variety of instruments can be employed in the process of the invention in order to generate the electromagnetic waves necessary to destabilize the cell juice: magnetrons, power grid tubes, klystrons, klystrodes, crossed-field amplifier, travelling wave tubes, and gyrotrons. One such instrument includes, but is not limited to high power magnetron. Conventional and industrial magnetrons operate at a frequency of 915 MHz and 2.45 GHz and can be employed. However at those frequencies undesirable heat is can be generated that can denature the cell juice composition. It is therefore advantageous to use electromagnetic waves operating at frequencies that are substantial higher than the frequencies of conventional or industrial magnetrons, which allows for destabilization of the cell juice without undesirable denaturing due to heat generation. This frequency is typically above the frequency of conventional microwave magnetrons, i.e., above 2.45 GHz, in another embodiment greater than 2.45 GHz and less than about 7 GHz; and in another embodiment from about 3 to about 6 GHz. During the destabilizing step of the invention the temperature of the cell juice is beneficially maintained below 40° C., in another embodiment below about 35° C., in another embodiment below about 30° C., in another embodiment below about 25° C., in another embodiment below about 20° C.

The freshly obtained membrane fraction commonly referred to in the art, as “protein-vitamin concentrate,” is a paste having intensive color and specific odor that is plant raw material source specific. The membrane fraction is represented predominantly by chloroplasts present in the green parts of plant or mostly by chromoplasts present in flowers. The composition of the membrane fraction includes predominantly phospholipids, membrane proteins, chlorophyll, nucleus, mitochondria and carotenoids.

### Process for Preparing Cytoplasm/Cytosole Fraction Derived Cosmetic Compositions Substantially-Free From Membrane Fractions

The present invention also relates to a method for preparing the cytoplasm/cytosole fraction derived cosmetic compositions substantially-free from membrane fractions exhibiting antioxidant activity, cell growth stimulation activity, or both antioxidant and cell growth stimulation activities. The method involves providing a cell juice that has been separated from a fresh plant biomass, as already described above with respect to the Membrane-Derived Cosmetic Composition. The plant cell juice is then treated under conditions effective to separate the plant cell juice into a membrane fraction and a cytoplasm/cytosole fraction.

The cytoplasm/cytosole fraction can then be optionally further processed under conditions effective to separate the cytoplasm/cytosole fraction into its component parts, namely the cytoplasm fraction and a cytosole fraction. The cytoplasm fraction includes predominantly white soluble proteins; in C3 plants, these proteins largely consist of the enzyme ribulose-1,5biphosphate carboxylase oxygenase. The cytosole fraction contains low molecular weight soluble components. Cytosole fraction is refined under conditions effective to yield a cell serum fraction having antioxidant activity, cell growth stimulation activity, or both antioxidant and cell growth stimulation activities. The cell serum fraction is stabilized under conditions effective to yield a stable bioactive botanical cosmetic composition exhibiting antioxidant activity, cell growth stimulation activity, or both antioxidant and cell growth stimulation activities as described for example, in U.S. Pat. Nos. 7,442,391; 7,473,435; 7,537,791; 8,043,635; 8,101,212 and 8,277,852.

The plant cell juice may be obtained from all types of plants. Examples of suitable plants that may be used as sources of fresh plant biomass in the present include, without limitation, plants from the following families: Laminariaceae, Cladophoraceae, Fabaceae, Theaceae, Asteraceae, Lamiaceae, Liliaceae, Poaceae and Moraceae. In particular, examples of specific plants that have been tested and found appropriate as fresh plant biomass sources include Macrocystis pyrifera, Chaetomorpha basiretorsa, Medicago sativa, Trifolium pratense, Citrus limon, Glycine max, Camellia sinensis, Calendula officinalis, Tanacetum parthenium, Chamomilla recutita, Lavandula angustifolia, Salvia officinalis, Nelumbo nucifera, Lilium bulbiferum, Avena sativa and Hordeum vulgare. Various parts of the plants may be used. For example, the stems and leaf tissue may be used for many types of plants. For other plants, the flowers may be used as sources of plant cell juice for use in the present invention. For example, one embodiment of the present invention uses flower tissue of Calendula officinalis for the separation of the plant cell juice. In another embodiment, the leaf and stem tissue is used.

The quantitative criteria to evaluate the complete separation of cytoplasm fraction is the absence of detectable levels of high molecular weight proteins and/or the absence of ribulose-1,5-biphosphate carboxylase oxygenase in cytosole fraction.

The cytosole fraction is clear liquid which has a slight yellow color and slight characteristic odor. In several hours, the unstable cytosole fraction is irreversibly transformed into dark brown color suspension containing heavy precipitate and strong non-characteristic odor. As a result, cytosole fraction cannot be used as a cosmetic ingredient. The described procedure that follows allows for the refinement of cytosole fraction to yield stable and active serum fraction which is stable cosmetic ingredients. This is accomplished by removing from cytosole fraction the major components responsible for the irreversible transformations that lead to generation of unwanted precipitate and deterioration of color and odor. This procedure includes: pH adjustment, heat treatment, cooling, vacuum filtration, and stabilization as described in U.S. Pat. Nos. 7,442,391, 8,101,212, and 8,277,852, which are all incorporated herein by reference.

After the cell serum fraction is produced, it is then subjected to the stabilizing step to yield the Serum-Derived Cosmetic Composition. In one embodiment, the stabilizing step involves incubating the cell serum fraction in a mixture of at least one preservative and at least one antioxidant to yield a stabilized cell serum fraction. Suitable preservatives for use in the present invention include, for example, potassium sorbate, sodium benzoate, sodium methyl paraben, and citric acid. An example of a suitable antioxidant for use in the present invention is sodium metabisulfite.

In one embodiment, the invention utilizes an isolated bioactive fraction derived from a Theacea plant. As used herein, the term “isolated bioactive fraction” is meant to include fractions that are isolated from a Theacea plant (e.g., fresh biomass of a Theacea plant) that has not undergone any conventional tea processing (e.g., heat treatment, oxidation, fermentation, drying). More particularly, Theacea plant fractions prepared in accordance with the present invention are unique in that have not undergone any substantial fermentation and are thus substantially free of the byproducts of fermentations including polyphenols. More particularly, the Theacea plant fractions of the invention are substantially free of polyphenols. The phrase “substantially free of polyphenols” is intended to mean that the plant fractions of the invention contain less than about 10% by weight polyphenols based on the dry weight of the plant fraction material, in another embodiment less than about 8% by weight polyphenols, in another embodiment less than about 6% by weight polyphenols, in another embodiment less than about 5% by weight polyphenols, in another embodiment less than about 4% by weight polyphenols, in another embodiment less than about 3% by weight polyphenols, in another embodiment less than about 2% by weight polyphenols, in another embodiment less than about 1% by weight polyphenols, in another embodiment less than about 0.5% by weight polyphenols, in another embodiment less than about 0.2% by weight polyphenols, and in yet another embodiment less than about 0.1% by weight polyphenols. In another embodiment, the fractions of the invention have undergone no fermentation and have no measurable polyphenols. Suitable isolated bioactive fractions can include, without limitation, a cell walls fraction, a cell walls fraction extract, a membrane fraction, a membrane fraction extract, a cytoplasm fraction, a cytoplasm fraction extract, a cell juice serum, and/or combinations thereof.

The bioactive compositions and bioactive fractions can have various catechin profiles and total catechin content amounts, as defined below, and as determined using conventional catechin diagnostic methods well known in the art. As used herein, the term “catechin” generally refers to all catechins, including, but not limited to, the following specific types of catechins: (i) (−)-epigallocatechin (see CAS No. 970-74-1, which is hereby incorporated by reference in its entirety); (ii) (+)-catechin (see CAS No. 7295-85-4, which is hereby incorporated by reference in its entirety); (iii) (−)-epicatechin (see CAS No. 490-46-0, which is hereby incorporated by reference in its entirety); (iv) (−)-epigallocatechin gallate (see CAS No. 989-51-5, which is hereby incorporated by reference in its entirety); (v) (−)-gallocatechin gallate (see CAS No. 4233-96-9, which is hereby incorporated by reference in its entirety); and (vi) (−)-epicatechin gallate (see CAS No. 1257-08-5, which is hereby incorporated by reference in its entirety). “Total catechin content” (as used herein) refers to the combined content level of all catechins contained in a particular bioactive composition or bioactive fraction, and is not meant to be limited to the content levels of just the specific types of catechins listed herein above. As used herein, the term “catechin content profile” is used to describe the amounts of selected catechins contained in a particular bioactive composition or bioactive fraction of the present invention.

In one embodiment of the bioactive composition of the present invention, the bioactive fraction can be a cell walls fraction.

In one embodiment of the bioactive composition of the present invention, the bioactive fraction can be a cell walls fraction extract. In a specific embodiment of the present invention, the cell walls fraction extract can have a total catechin content of between about 2.1 and about 4.5 milligrams per gram of dry matter, particularly between about 2.6 and about 4.0 milligrams per gram of dry matter, and more particularly between about 3.0 and about 3.6 milligrams per gram of dry matter. In another specific embodiment, the cell walls fraction extract can have a catechin content profile as follows: (i) between about 2.0 and about 3.0 milligrams of (+)-catechin per gram of dry matter of the cell walls fraction extract; (ii) between about 0.005 and about 0.02 milligrams of (−)-epicatechin per gram of dry matter of the cell walls fraction extract; (iii) between about 0.005 and about 0.02 milligrams of (−)-epigallocatechin gallate per gram of dry matter of the cell walls fraction extract; and (iv) between about 0.003 and about 0.01 milligrams of (−)-epicatechin gallate per gram of dry matter of the cell walls fraction extract. More particularly, the cell walls fraction extract can have a catechin content profile as follows: (i) between about 2.2 and about 2.7 milligrams of (+)-catechin per gram of dry matter of the cell walls fraction extract; (ii) between about 0.01 and about 0.015 milligrams of (−)-epicatechin per gram of dry matter of the cell walls fraction extract; (iii) between about 0.01 and about 0.015 milligrams of (−)-epigallocatechin gallate per gram of dry matter of the cell walls fraction extract; and (iv) between about 0.005 and about 0.007 milligrams of (−)-epicatechin gallate per gram of dry matter of the cell walls fraction extract.

In one embodiment of the bioactive composition of the present invention, the bioactive fraction can be a membrane fraction.

In one embodiment of the bioactive composition of the present invention, the bioactive fraction can be a membrane fraction extract. In a specific embodiment of the present invention, the membrane fraction extract can have a total catechin content of between about 15.0 and about 30.5 milligrams per gram of dry matter, particularly between about 18.0 and about 27.5 milligrams per gram of dry matter, and more particularly between about 21.0 and about 24.5 milligrams per gram of dry matter. In another specific embodiment, the membrane fraction extract can have a catechin content profile as follows: (i) between about 1.7 and about 3.3 milligrams of (−)-epigallocatechin per gram of dry matter of the membrane fraction extract; (ii) between about 6.1 and about 10.2 milligrams of (+)-catechin per gram of dry matter of the membrane fraction extract; (iii) between about 0.3 and about 1.1 milligrams of (−)-epicatechin per gram of dry matter of the membrane fraction extract; (iv) between about 6.2 and about 12.5 milligrams of (−)-epigallocatechin gallate per gram of dry matter of the membrane fraction extract; (v) between about 0.007 and about 0.03 milligrams of (−)-gallocatechin gallate per gram of dry matter of the membrane fraction extract; and (vi) between about 1.3 and about 3.3 milligrams of (−)-epicatechin gallate per gram of dry matter of the membrane fraction extract. More particularly, the membrane fraction extract can have a catechin content profile as follows: (i) between about 2.0 and about 3.0 milligrams of (−)-epigallocatechin per gram of dry matter of the membrane fraction extract; (ii) between about 7.0 and about 9.0 milligrams of (+)-catechin per gram of dry matter of the membrane fraction extract; (iii) between about 0.5 and about 0.9 milligrams of (−)-epicatechin per gram of dry matter of the membrane fraction extract; (iv) between about 8.0 and about 10.0 milligrams of (−)-epigallocatechin gallate per gram of dry matter of the membrane fraction extract; (v) between about 0.01 and about 0.02 milligrams of (−)-gallocatechin gallate per gram of dry matter of the membrane fraction extract; and (vi) between about 1.8 and about 2.8 milligrams of (−)-epicatechin gallate per gram of dry matter of the membrane fraction extract.

In one embodiment of the bioactive composition of the present invention, the bioactive fraction can be a cytoplasm fraction.

In one embodiment of the bioactive composition of the present invention, the bioactive fraction can be a cytoplasm fraction extract.

In one embodiment of the bioactive composition of the present invention, the bioactive fraction can be a cell juice serum. In a specific embodiment, the cell juice serum can have a total catechin content of between about 8.0 and about 20.0 milligrams per gram of dry matter, particularly between about 10.0 and about 18.0 milligrams per gram of dry matter, and more particularly between about 12.0 and about 16.0 milligrams per gram of dry matter. In another specific embodiment, the cell juice serum can have a catechin content profile as follows: (i) between about 2.1 and about 4.4 milligrams of (−)-epigallocatechin per gram of dry matter of the cell juice serum; (ii) between about 4.2 and about 8.6 milligrams of (+)-catechin per gram of dry matter of the cell juice serum; (iii) between about 0.2 and about 2.0 milligrams of (−)-epicatechin per gram of dry matter of the cell juice serum; (iv) between about 1.2 and about 3.2 milligrams of (−)-epigallocatechin gallate per gram of dry matter of the cell juice serum; (v) between about 0.01 and about 0.1 milligrams of (−)-gallocatechin gallate per gram of dry matter of the cell juice serum; and (vi) between about 0.2 and about 1.3 milligrams of (−)-epicatechin gallate per gram of dry matter of the cell juice serum. More particularly, the cell juice serum can have a catechin content profile as follows: (i) between about 3.0 and about 3.5 milligrams of (−)-epigallocatechin per gram of dry matter of the cell juice serum; (ii) between about 5.0 and about 7.0 milligrams of (+)-catechin per gram of dry matter of the cell juice serum; (iii) between about 0.7 and about 1.5 milligrams of (−)-epicatechin per gram of dry matter of the cell juice serum; (iv) between about 1.7 and about 2.7 milligrams of (−)-epigallocatechin gallate per gram of dry matter of the cell juice serum; (v) between about 0.03 and about 0.07 milligrams of (−)-gallocatechin gallate per gram of dry matter of the cell juice serum; and (vi) between about 0.5 and about 1.0 milligrams of (−)-epicatechin gallate per gram of dry matter of the cell juice serum.

In one embodiment, fresh biomass of Theacea plants can be used to isolate the bioactive compositions of the present invention. The fresh biomass can be taken from Theacea plants that are of the Camellia and/or Eurya genera. Suitable species of the Camellia genus for use in the present invention can include, without limitation, Camellia sinensis, Camellia japonica, Camellia reticulate, and Camellia sasanqua. Suitable species of the Eurya genus for use in the present invention can include, without limitation, Eurya sandwicensis.

The bioactive composition of the present invention can further include a stabilizing agent. Suitable stabilizing agents are those that are commonly used in the art. Particular suitable stabilizing agents can include, without limitation, an emulsifier, a preservative, an anti-oxidant, a polymer matrix, and/or mixtures thereof.

In one aspect of the present invention, the bioactive fraction can have modulatory activity on at least one mammal cell function. Such modulatory activity can include, for example, cell growth inhibition activity, cell growth stimulation activity, enzyme secretion activity, enzyme inhibition activity, anti-oxidant activity, UV-protection activity, anti-inflammatory activity, wound healing activity, and/or combinations of these activities. With respect to cell growth inhibition activity, such activity can involve growth inhibition of cancer cells. Suitable cancer cells that can be inhibited to grow by the bioactive fractions of the present invention can include, without limitation, breast cancer cells and/or colon cancer cells. The described cell growth inhibition activity can also include growth inhibition of leukemia cells. Suitable leukemia cells that can be inhibited to grow by the bioactive fractions of the present invention can include, without limitation, monocytic leukemia cells.

In another embodiment, the bioactive composition can be effective in inhibiting unwanted hyper-proliferation or hypo-proliferation of skin cells and/or inhibiting unwanted uncoordinated enzyme activities or enzyme secretion processes in the skin cells.

In another embodiment, the bioactive composition of the present invention can further include a delivery system for systemic or topical administration that are commonly used in the art.

The present invention also relates to a bioactive topical formulation suitable for topical application to a mammal. In one embodiment, the bioactive topical formulation includes a topically effective amount of the bioactive composition of the present invention.

The bioactive topical formulation can further include a topically acceptable carrier. Suitable topically acceptable carriers can include, without limitation, a hydrophilic cream base, a hydrophilic lotion base, a hydrophilic surfactant base, a hydrophilic gel base, a hydrophilic solution base, a hydrophobic cream base, a hydrophobic lotion base, a hydrophobic surfactant base, a hydrophobic gel base, and/or a hydrophobic solution base. In one embodiment, the bioactive composition can be present in an amount ranging from between about 0.001 percent and about 90 percent of the total weight of the bioactive topical formulation.

The present invention also relates to a method for inhibiting inflammatory activity in skin tissue of a mammal. This method involves providing the bioactive composition according to the present invention. The method further involves applying the bioactive composition to the skin tissue in an amount effective to inhibit inflammatory activity in the skin tissue. In one embodiment of this method, the bioactive composition can further include a stabilizing agent (suitable examples of which are as described herein). In another embodiment of this method, the bioactive composition can further include a topically acceptable carrier (suitable examples of which are as described herein).

### EXAMPLES

### Summary of the Examples

The processes described in, for example, U.S. Pat. Nos. 7,442,391; 7,473,435; 7,537,791; 8043635, 8,101,212; 8,277,852 and 8,318,220, all incorporated herein by reference, uniquely preserves biological activities in natural ingredients. This invention describes a novel approach to mitigate surfactant-induced skin irritation and inflammation using unique combinations of bioactive compositions and surfactants. In this invention, Zeta Fraction™ technology derived natural fractions, such as Recentia® CS, Recentia® CL, and Recentia® TP, were evaluated in in vitro surfactant-induced skin cell inflammation/irritation model to determine if these ingredients have potential to mild impact of personal care and cleaning products. Cultured human epidermal keratinocytes (HEK) were treated with surfactants to induce release of key inflammatory cytokine interleukin (IL-1α) and key chemokine interleukin (IL-8). Cytotoxicity was evaluated with LDH assay. Based on cytotoxicity data and abilities to induce release of IL-1α, six representatives of four different classes of surfactants were ranked. A nonionic ethoxylated alcohol was the mildest surfactant and was able to induce release of IL-1α but not IL-8. It was found, that Recentia® CS was a potent inhibitor of IL-1α and IL-8 in HEK treated with either SDS or ethoxylated alcohol. Activity of Recentia® CS was comparable to aspirin (positive control for IL-1α) for inhibition of IL-1α induced by either SDS or ethoxylated alcohol. Aspirin was ineffective for inhibition of IL-1α induced by ethoxylated alcohol. Importantly, Recentia® CS inhibited both SDS-induced and basal level IL-8 in keratinocytes, while SB203580 (known positive control for IL-8) inhibited SDS-induced IL-8 only. Traditional green tea and black tea preparations obtained from the same cultivar as Recentia® CS failed to inhibit IL-1α and IL-8 induced by either SDS or ethoxylated alcohol. In addition, both Citrus limon (Recentia® CL) and Trifolium pratense (Recentia® TP) also inhibited SDS-induced IL-1α to a lesser extent. In conclusion, the unique combination of surfactants with Zeta Fraction™ technology derived natural fractions including, without limitation, Camellia sinensis (Recentia® CS), Citrus limon (Recentia® CL), and Trifolium pratense (Recentia® TP), provides a novel approach to mitigate surfactant-induced skin irritation and inflammation responses and impart the mildness to the next generation of surfactant-containing products.

### Example 1

### Sodium Dodecyl Sulfate (SDS), as a Benchmark Anionic Surfactant, Dose-Dependently Induces Inflammatory Cytokine IL-1α and Chemokine IL-8 in Human Epidermal Keratinocyte (HEK) Culture

Sodium dodecyl sulphate (SDS) is an anionic surfactant widely used in personal care and cleansing products. SDS, which is a well-known inducer of experimental irritant contact dermatitis, has been shown to stimulate multiple cytokine release including IL-1α and chemokine IL-8 in epidermal skin cells (Craig et al., JID 115:292, 2000; and Chung et al., JID 117:647, 2001). To validate the cell culture model for evaluation of inflammatory response induced by surfactants, SDS was used as a benchmark anionic surfactant to induce inflammatory mediators including IL-1α and IL-8 in HEK.

The protocols for the growth and treatment of human primary epidermal keratinocyte (HEK) cultures are as follows. HEK and all cell culture supplies were obtained from Life Technologies Co. (Carlsbad, Calif.). The cells were maintained in keratinocyte growth medium (KGM), which contains keratinocyte basal medium 154 (M154) and human keratinocyte growth supplements (HKGS). They were grown at 37° C. in an atmosphere of 5% (v/v) CO₂ and used between passages from 2 to 4. For the treatment, keratinocytes were trypsinized by Trypsin 0.025%/EDTA 0.01% in Phosphate Buffered Saline, seeded in 96-well plates, and grown in KGM to about 80% confluence. The cells were then exposed to SDS (Sigma-Aldrich Co., St. Louis, Mo.) at various concentrations for about 16 hours. After incubation, the supernatants were collected, and IL-1α and IL-8 were quantified using Quantikine® ELISA (R&D Systems, Minneapolis, Minn.). Results were presented as the mean±standard deviation. IC₅₀ was calculated using sigmoidal curve fitting with SigmaPlot Version 10.0 (Systat Software). LDH (lactate dehydrogenase) assay (G-Biosciences, St. Louis, Mo.) was performed on all the supernatants to assess cytotoxicity.

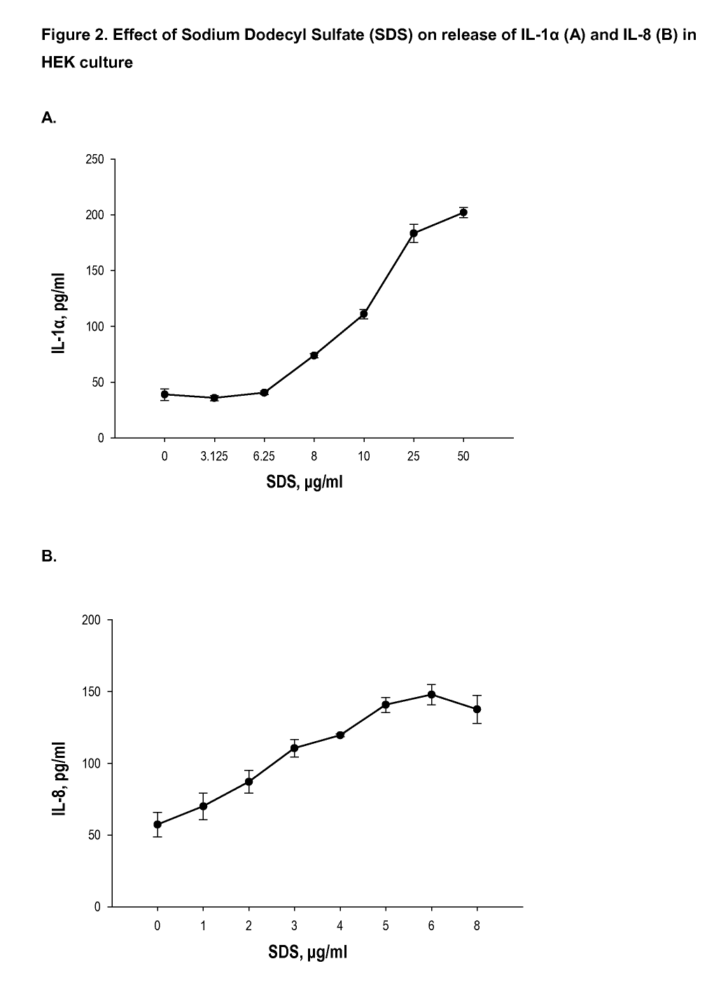

As shown in the FIG. 2A and 2B, SDS induced dose-dependent release of inflammatory cytokine IL-1α (A) and chemokine IL-8 (B) after incubation with HEK for 16 hours. SDS at 25 μg/ml showed significant (p<0.001) induction of IL-1α. SDS at 6 μg/ml showed significant (p<0.001) induction of IL-8. These concentrations were used as the induction doses in the rest of the experiments.

### Example 2

### Comparison of the Mildness of Selected Commercially Available Surfactants Including SDS Based on Cytotoxicity and Release of IL-1α

To evaluate different classes of surfactants for their ability to induce skin cell damage and inflammatory response, six representatives of four different classes of commercially available surfactants were ranked based on cytotoxicity data from LDH assay and induction of primary inflammatory cytokine IL-1α release in HEK.

The protocols for the growth of HEK are described under Example 1. In place of SDS, a range of concentrations of six different surfactants was used.

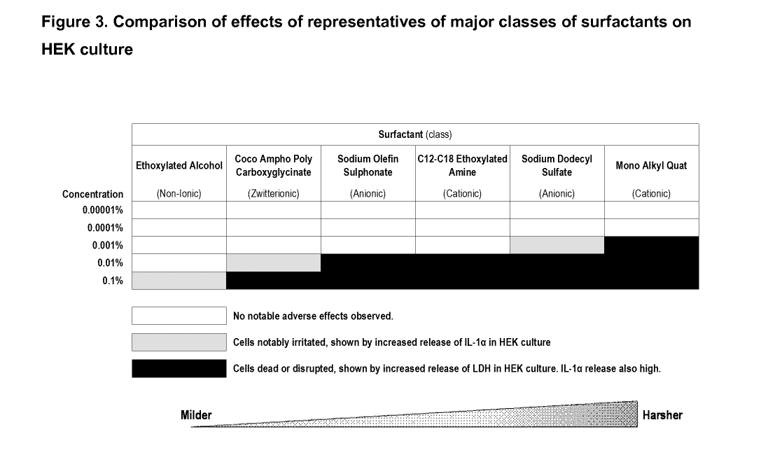

As shown in FIG. 3, the tested surfactants are listed in the order of increasing harshness: Ethoxylated Alcohol, Coco Ampho Poly Carboxyglycinate, Sodium Olefin Sulphonate, C12-C18 Ethoxylated Amine, Sodium Dodecyl Sulfate, Mono Alkyl Quat. The concentrations given are percent in HEK cultivation medium (KGM), assuming “as supplied” surfactant material as 100%.

### Example 3

### An Ethoxylated Alcohol, as an Example of Nonionic Surfactant, Induces Dose-Dependent Release of IL-1α, but not IL-8, in HEK

Based on the mildness ranking of the surfactants in Example 2, ethoxylated alcohol was ranked as the mildest surfactant of those tested. It is widely used in general purpose and high pressure cleaning formulations, and in household hard surface cleaning. Its functions include cleanser, emulsifier, micro emulsion, and wetting. Therefore, we tested the ethoxylated alcohol as a representative mild nonionic surfactant to induce inflammatory cytokine IL-1α and chemokine IL-8 in HEK.

The protocols for the growth of HEK are described under Example 1. Instead of SDS, the keratinocytes were exposed to a range of concentrations of ethoxylated alcohol for about 16 hours.

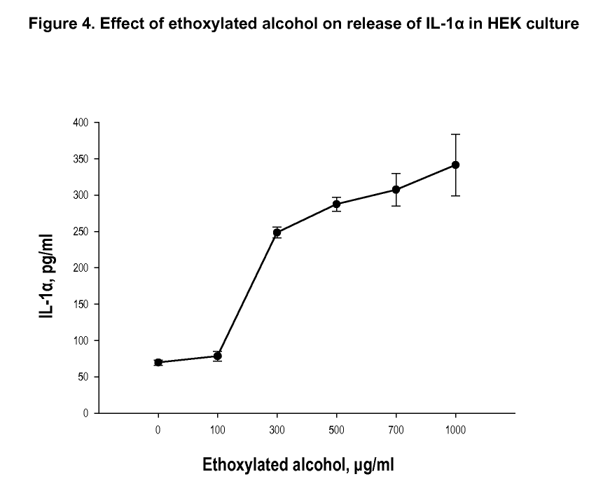

As shown in the FIG. 4, the ethoxylated alcohol at 500-1000 μg/ml demonstrated significant (p<0.001) induction of IL-1α. This concentration range was used as induction doses in the rest of the experiments.

It should be noted, that no significant induction of chemokine IL-8 by ethoxylated alcohol was observed, while SDS induced both IL-1α and IL-8 significantly. This result correlates to the findings in Example 2, in which we have ranked surfactants from different classes and have shown that ethoxylated alcohol was the mildest surfactant, while SDS was close to the harshest surfactant.

### Example 4

### Serum Fraction of Camellia sinensis (i.e. Recentia® CS) inhibits SDS-induced IL-1α and IL-8 Dose-Dependently, as Compared to Anti-Inflammatory Benchmark Agents

Serum Fraction of Camellia sinensis (Recentia® CS) is obtained from fresh Camellia sinensis (tea plant) by process described in, for example, U.S. Pat. Nos. 7,473,435, 8,043,635, and 8,318,220. It delivers multiple benefits for free-radical scavenging properties, anti-inflammatory benefits, and photostabilization activity. Therefore, we compared Recentia® CS to anti-inflammatory benchmark agents (positive controls) for mitigating the release of inflammatory cytokine IL-1α and chemokine IL-8 in HEK.

The protocols for the growth of HEK are described under Example 1. Treatment was conducted by incubation of HEK with SDS (25 μg/ml for induction of IL-1α or 6 μg/ml for induction of IL-8 as determined in Example 1) for 16 hours in presence of a range of concentrations of Recentia® CS, or Aspirin, or SB203580.

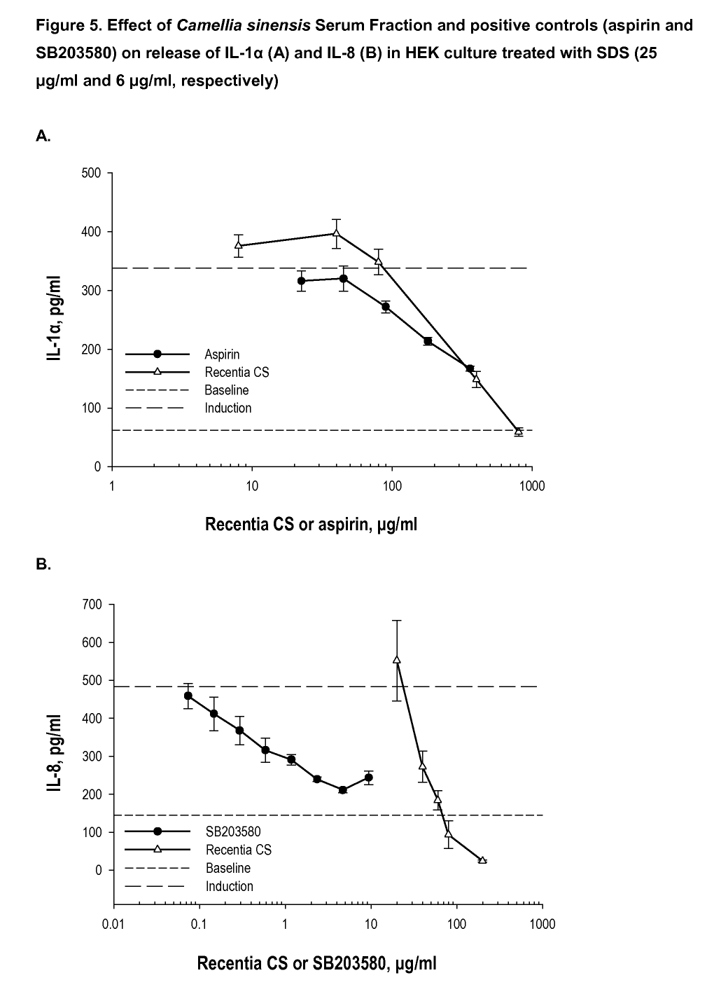

FIG. 5A shows that Aspirin, a well-known anti-inflammatory benchmark agent (Reviewed by Jue DM, et al. 1999; 14 (3): 231-8), demonstrated dose-dependent inhibition of IL-1α release induced by SDS (25 μg/ml) in HEK. Estimated IC₅₀ of Aspirin was about 230 μg/ml. Recentia® CS was compared to aspirin for mitigating SDS-induced release of the inflammatory cytokine IL-1α. It was found that Recentia® CS is an effective inhibitor of SDS-induced release of IL-1α. Estimated IC₅₀ of Recentia CS was about 310 μg/ml, suggesting the comparable potency as that of Aspirin.

In FIG. 5A, “Baseline” refers to the concentration of IL-1α in supernatant of untreated HEK. “Induction” refers to the concentration of IL-1α in supernatant of HEK treated with 25 μg/ml of SDS, which is the induction dose determined in Example 1. Due to variations inherent in biological test systems such as HEK cultures produced from cells harvested from different donors or different donor body locations, response of HEK as shown by concentrations of cytokines (e.g. IL-1α) and chemokines (e.g. IL-8) may vary between experiments, particularly the “Baseline” and “Induction” concentrations.

Importantly, Aspirin did not inhibit SDS-induced release of inflammatory chemokine IL-8. We thus evaluated another anti-inflammatory benchmark agent, SB203580, a p38 MAPK kinase inhibitor (Reviewed by Lee JC et al. Immunopharmacology 2000; 47, 185-201). As shown in FIG. 5B, SB203580 demonstrated dose-dependent inhibition of IL-8 release induced by SDS (6 μg/ml) with estimated IC₅₀ of about 0.70 μg/ml. When we compared to SB203580, Recentia® CS showed dose-dependent inhibition of IL-8 induced by SDS (6 μg/ml) with estimated IC₅₀ of about 34 μg/ml. It was especially important to find that Recentia CS inhibited both SDS-induced and basal level non-induced IL-8 release, while SB203580 inhibited SDS-induced IL-8 only.

In FIG. 5B, “Baseline” refers to the concentration of IL-8 in supernatant of untreated HEK. “Induction” refers to the concentration of IL-8 in supernatant of HEK treated with 6 μg/ml of SDS, which is the induction dose determined in Example 1. Due to variations inherent in biological test systems such as HEK cultures produced from cells harvested from different donors or different donor body locations, response of HEK as shown by concentrations of cytokines (e.g. IL-1α) and chemokines (e.g. IL-8) may vary between experiments, particularly the “Baseline” and “Induction” concentrations.

### Example 5

### Serum Fraction of Camellia sinensis (i.e. Recentia® CS) Inhibits Ethoxylated Alcohol-Induced Release of IL-1α Dose-Dependently

We further evaluated Aspirin for inhibition of the IL-1α release induced by the ethoxylated alcohol.

The protocols for the growth of HEK are described under Example 1. Treatment was conducted by incubation of HEK with ethoxylated alcohol (1000 μg/ml for induction of IL-1α as determined in Example 3) for 16 hours in presence of a range of concentrations of Recentia® CS, or Aspirin.

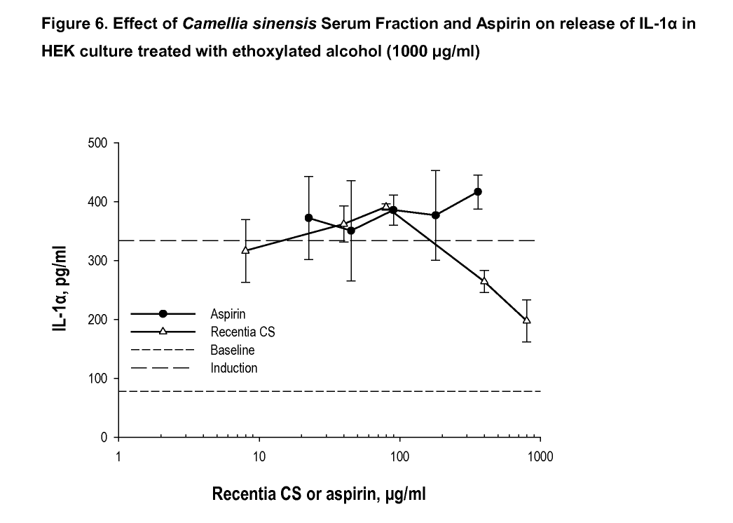

As shown in FIG. 6, Aspirin failed to inhibit IL-1α release induced by the ethoxylated alcohol. Surprisingly, Recentia® CS effectively inhibited ethoxylated alcohol-induced IL-1α release dose-dependently with estimated IC₅₀ of about 560 μg/ml in HEK. This data clearly indicates that Recentia® CS can mitigate IL-1α release induced by different classes of surfactants.

In FIG. 6, “Baseline” refers to the concentration of IL-1α in supernatant of untreated

HEK. “Induction” refers to the concentration of IL-1α in supernatant of HEK treated with 1000 μg/ml of ethoxylated alcohol, which is the induction dose determined in Example 3. Due to variations inherent in biological test systems such as HEK cultures produced from cells harvested from different donors or different donor body locations, response of HEK as shown by concentrations of cytokines (e.g. IL-1α) and chemokines (e.g. IL-8) may vary between experiments, particularly the “Baseline” and “Induction” concentrations.

### Example 6

### Conventional Green Tea and Black Tea Preparations from the Source Identical to Serum Fraction of Camellia sinensis (i.e. Recentia® CS), Have No Inhibition of IL-1α and IL-8 Induced by SDS

To demonstrate superior activity of Recentia® CS for mitigating the release of the inflammatory cytokine IL-1α and IL-8 induced by SDS, we did a side by side fair comparison of conventional preparations of commercially available green tea and black tea from source identical to material used for obtaining Recentia® CS used in this invention. The conventional tea preparations were made in accordance with manufacturer-suggested methods described below.

Conventional green tea preparation was obtained from the same cultivar of tea plant grown and harvested under the same conditions and at the same time as that used for obtaining Serum Fraction of Camellia sinensis (Recentia® CS). 2 grams of “Island Green Premium Tea” from Charleston Tea Plantation, Wadmalaw Island, S.C., USA, were steeped in 200 grams of deionized water at 85° C. on a magnetic stirrer for 2 minutes. Resulting liquid was strained, allowed to cool to room temperature, divided into aliquots in cryogenic vials and stored at −30° C. for further experimental use.

Conventional black tea preparation was obtained from the same cultivar of tea plant grown and harvested under the same conditions and at the same time as that used for obtaining Serum Fraction of Camellia sinensis (Recentia® CS). 2 grams of “Limited Edition Exceptional Quality 100% First Flush Loose Tea” from Charleston Tea Plantation, Wadmalaw Island, S.C., USA, were steeped in 200 grams of deionized water at 99° C. on a magnetic stirrer for 4 minutes. Resulting liquid was strained, allowed to cool to room temperature, divided into aliquots in cryogenic vials and stored at −30° C. for further experimental use.

Serum Fraction of Camellia sinensis (Recentia® CS) was obtained from fresh

Camellia sinensis (tea plant) by process described in, for example, U.S. Pat. Nos. 7,473,435, 8,043,635, and 8,318,220.

The protocols for the growth of HEK are described under Example 1. Treatment was conducted by incubation of HEK with SDS (25 μg/ml for induction of IL-1α or 6 μg/ml for induction of IL-8 as determined in Example 1) for 16 hours in presence of a range of concentrations of Recentia® CS, or conventional green tea preparation, or conventional black tea preparation.

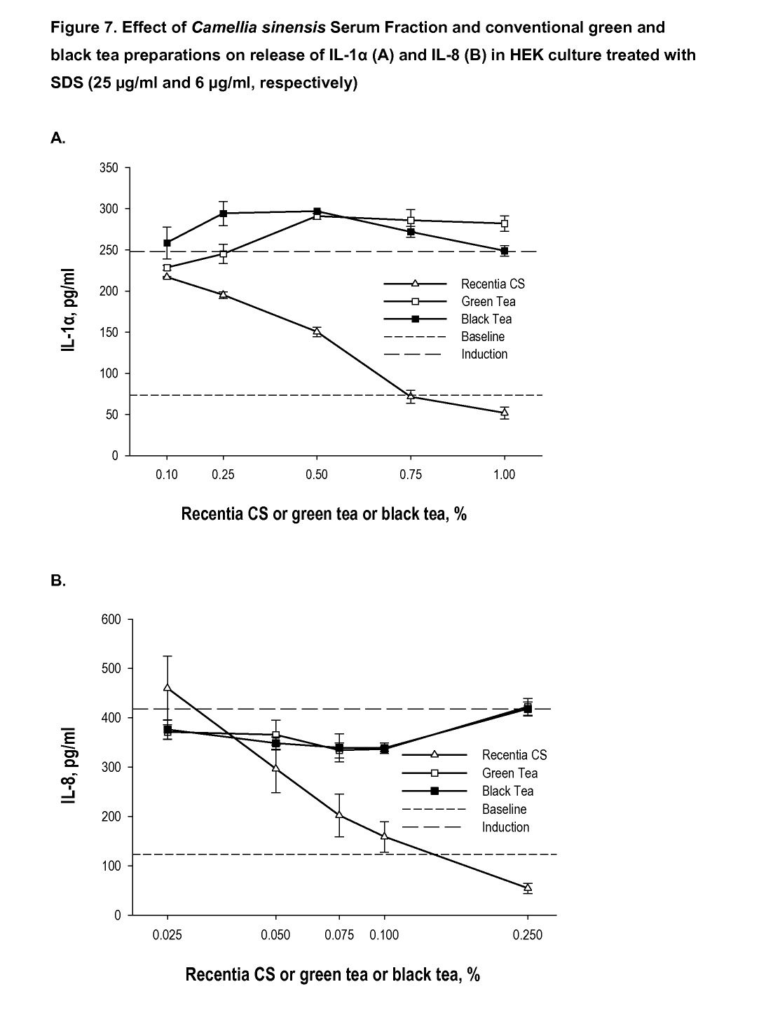

As shown in the FIG. 7A-B, Recentia® CS dose-dependently inhibited SDS-induced release of IL-1α (A) with estimated IC₅₀ of about 0.39% (with notable inhibition starting at the concentration of about 0.1%), and release of IL-8 (B) with estimated IC₅₀ of about 0.043% (with notable inhibition starting at the concentration of about 0.04%). While the highest concentration of Recentia® CS tested was 1%, this should not be construed as the high limit of concentration range effective for inhibition. The concentrations of Recentia® CS, or conventional green tea preparation, or conventional black tea preparation indicated in FIG. 7A-B are percent by volume in HEK growth medium, assuming “as is” material as 100%. However, neither green tea nor black tea at the same concentrations showed notable inhibition. The data indicate that Recentia® CS, processed by patented Zeta Fraction™ technology, has unique and diverse composition of natural constituents that exhibit superior multifunctional benefits than preparations of green tea and black tea.

In FIG. 7A, “Baseline” refers to the concentration of IL-1α in supernatant of untreated HEK. “Induction” refers to the concentration of IL-1α in supernatant of HEK treated with 25 μg/ml of SDS, which is the induction dose determined in Example 1.

In FIG. 7B, “Baseline” refers to the concentration of IL-8 in supernatant of untreated HEK. “Induction” refers to the concentration of IL-8 in supernatant of HEK treated with 6 μg/ml of SDS, which is the induction dose determined in Example 1. Due to variations inherent in biological test systems such as HEK cultures produced from cells harvested from different donors or different donor body locations, response of HEK as shown by concentrations of cytokines (e.g. IL-1α) and chemokines (e.g. IL-8) may vary between experiments, particularly the “Baseline” and “Induction” concentrations.

### Example 7

### Conventional Green Tea and Black Tea Prepared from the Source Identical to Serum Fraction of Camellia sinensis (i.e. Recentia® CS), Have No Inhibition of IL-1α Induced by Ethoxylated Alcohol

Recentia® CS was compared to conventional green tea and black tea preparations obtained from the same source for mitigating ethoxylated alcohol-induced secretion of the inflammatory cytokine IL-1α.

Conventional green tea and conventional black tea preparations were obtained as described in Example 6. In particular, the preparations used for Example 6 and Example 7 are aliquots of identical material.

The protocols for the growth of HEK are described under Example 1. Treatment was conducted by incubation of HEK with ethoxylated alcohol (500 μg/ml for induction of IL-1α, as determined in Example 3) for 16 hours in presence of a range of concentrations of Recentia® CS, or conventional green tea preparation or conventional black tea preparation.

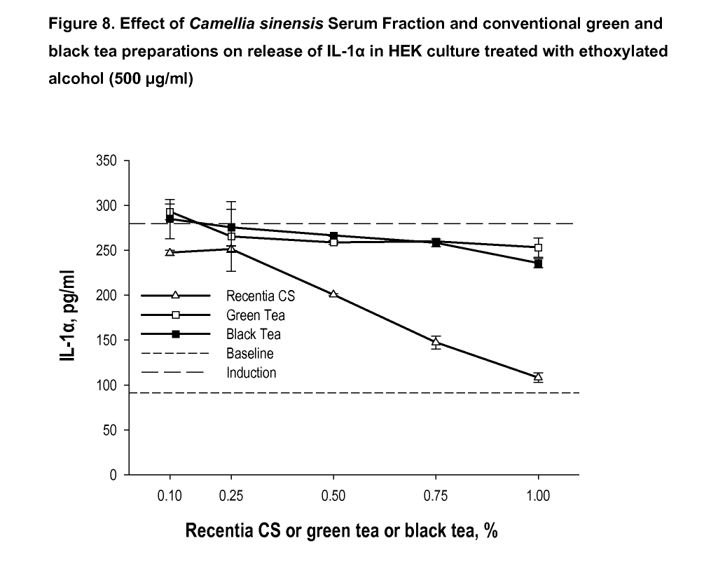

As shown in the FIG. 8, Recentia® CS dose-dependently inhibited ethoxylated alcohol-induced release of IL-1α with estimated IC₅₀ of about 0.70% (with notable inhibition starting at the concentration of about 0.3%). However, neither green tea nor black tea at the same concentrations showed notable inhibition. While the highest concentration of Recentia® CS tested was 1%, this should not be construed as the high limit of concentration range effective for inhibition. The data indicate that Recentia® CS, processed by patented Zeta Fraction™ technology, has unique and diverse composition of natural constituents that exhibit superior multifunctional benefits than preparations of green tea and black tea.

In FIG. 8, “Baseline” refers to the concentration of IL-1α in supernatant of untreated HEK. “Induction” refers to the concentration of IL-1α in supernatant of HEK treated with 500 μg/ml of ethoxylated alcohol, which is the induction dose determined in Example 3. Due to variations inherent in biological test systems such as HEK cultures produced from cells harvested from different donors or different donor body locations, response of HEK as shown by concentrations of cytokines (e.g. IL-1α) and chemokines (e.g. IL-8) may vary between experiments, particularly the “Baseline” and “Induction” concentrations.

### Example 8

### Serum Fractions of Citrus limon (Recentia® CL) and Trifolium pratense (Recentia® TP) Inhibit SDS-Induced IL-1a Dose-Dependently

Citrus limon (Recentia® CL) and Trifolium pratense (Recentia® TP) are processed as described in, for example, U.S. Pat. Nos. 7,473,435, 8,043,635, and 8,318,220. They deliver multiple benefits for free-radical scavenging properties, anti-inflammatory benefits, and photo stabilization activity. Therefore, we evaluated Citrus limon (Recentia® CL) and Trifolium pratense (Recentia® TP) for mitigating SDS-induced secretion of the inflammatory cytokine IL-1α.

The protocols for the growth of HEK are described under Example 1. Treatment was conducted by incubation of HEK with SDS alcohol (25 μg/ml for induction of IL-1α, as determined in Example 1) for 16 hours in presence of a range of concentrations of Recentia® CL, or Recentia® TP.

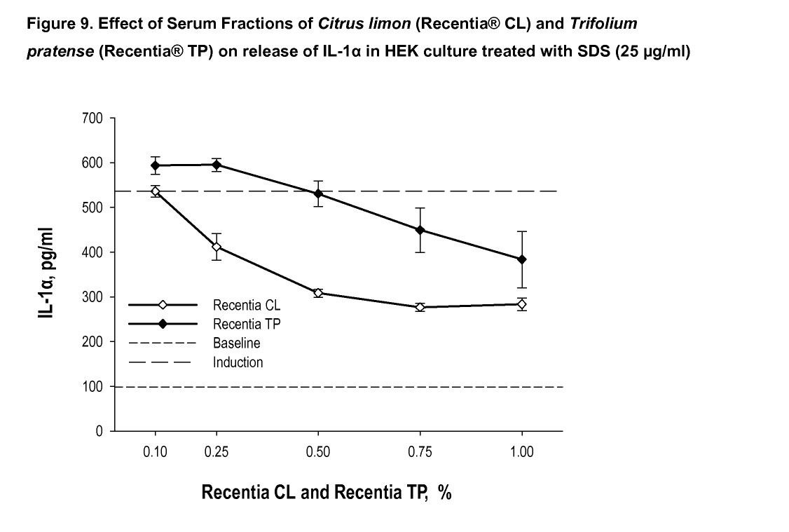

As shown in the FIG. 9, Citrus limon (Recentia® CL) dose-dependently inhibited IL-1α release induced by SDS in HEK, with estimated IC50 of about 0.46% (with notable inhibition starting at about 0.2%). Trifolium pratense (Recentia® TP) also dose-dependently mitigated SDS-induced release of the inflammatory cytokine IL-1α, with 1% inhibiting about 35% (with notable inhibition starting at about 0.75%) of SDS-induced release of IL-1α. While the highest concentration of Recentia® CL and Recentia® TP tested was 1%, this should not be construed as the high limit of concentration ranges effective for inhibition.

In FIG. 9, “Baseline” refers to the concentration of IL-1α in supernatant of untreated

HEK. “Induction” refers to the concentration of IL-1α in supernatant of HEK treated with 25 μg/ml of SDS, which is the induction dose determined in Example 1. Due to variations inherent in biological test systems such as HEK cultures produced from cells harvested from different donors or different donor body locations, response of HEK as shown by concentrations of cytokines (e.g. IL-1α) and chemokines (e.g. IL-8) may vary between experiments, particularly the “Baseline” and “Induction” concentrations.

In conclusion, the unique combination of surfactants with Zeta Fraction™ technology derived natural fractions including, without limitation, Camellia sinensis (Recentia® CS), Citrus limon (Recentia® CL), and Trifolium pratense (Recentia® TP), provides a novel approach to mitigate surfactant-induced skin irritation and inflammation responses and impart the mildness to the next generation of surfactant-containing products.

We claim:

1. A composition with reduced inflammation properties towards biological skin, said composition comprising at least one surfactant and an effective amount of a at least one biologically active plant fraction, wherein said biologically active plant fraction is a plant derived membrane fraction, cytoplasm fraction, cell juice serum, or a mixture or combination thereof, and wherein said plant fraction is substantially free from polyphenols.

2. The composition of claim 1 wherein said biologically active plant fraction is derived from Laminariaceae, Cladophoraceae, Fabaceae, Theaceae, Asteraceae, Lamiaceae, Liliaceae, Poaceae, Moraceae, Macrocystis pyrifera, Chaetomorpha basiretorsa, Medicago sativa, Trifolium pratense, Glycine max, Camellia sinensis, Citrus limon, Calendula officinalis, Tanacetum parthenium, Chamomilla recutita, Lavandula angustifolia, Salvia officinalis, Nelumbo nucifera, Lilium bulbiferum, Avena sativa, Hordeum vulgare, and combinations or mixtures thereof.

3. The composition of claim 12 wherein said biologically active plant fractions are derived from Camellia sinensis, Citrus limon, and Trifolium pratense.

4. The composition of claim 3 wherein said biologically active plant fractions are cell juice serum fractions.

5. The composition of claim 1, wherein the composition is a personal care product, wherein said product is a leave-on product selected from the group consisting of, cream, dressing, gel, lotion, ointment, liquid, a spray applicator, and combinations thereof, or a wash-off product selected from the group consisting of hand dishwashing detergent, liquid hand soap, bar soap, body wash, shampoo, general purpose cleanser, and combinations thereof.

6. The composition of claim 5 wherein said composition is a soap comprising at least one surfactant, and an effective amount of a biologically active plant fraction, wherein said biologically active plant fraction is a plant derived membrane fraction, cytoplasm fraction, cell juice serum, or mixtures or combination thereof.

7. The composition of claim 6 wherein said biologically active plant fraction is a cell juice serum fraction derived from Camellia sinensis, Citrus limon, and Trifolium pratense.

8. The composition of claim 5 wherein said composition is a skin cream comprising at least one surfactant, and an effective amount of a biologically active plant fraction, wherein said biologically active plant fraction is a plant derived membrane fraction, cytoplasm fraction, cell juice serum, or mixtures or combination thereof.

9. The composition of claim 6 wherein said biologically active plant fractions are cell juice serum fractions derived from Camellia sinensis, Citrus limon, and Trifolium pratense.

10. The composition of claim 1 wherein said plant fraction contains less than about 5% by weight polyphenols.

11. The composition of claim 3 wherein said plant fraction contains less than about 1% by weight polyphenols.

12. A method for reducing the inflammation of biological tissue said method comprising contacting said biological tissue with at least one bioactive plant fraction, wherein said bioactive plant fraction is a plant derived membrane fraction, cytoplasm fraction, cell juice serum, and combinations thereof, and wherein said plant fraction is substantially free of polyphenols.

13. The method of claim 12 wherein said inflammation is induced by a surface active compound.

14. The method of claim 13 wherein said surface active agent is a surfactant chosen from anionic, cationic, nonionic, zwitterionic, and combinations thereof.

15. The method of claim 12 wherein said plant fraction is derived from Camellia sinensis (Recentia® CS), Citrus limon (Recentia® CL), Trifolium pratense (Recentia® TP), or combinations and/or mixtures thereof.

16. The method of claim 15 wherein said plant fraction contains less that about 1% polyphenols based on the dry weight of said fraction.

17. The method of claim 16 wherein said fraction is serum faction.
